(19) United States
(12) Patent Application Publication
(10) Pub. No.: US 2022/0289811 A1
(43) Pub. Date: Sep. 15, 2022

(54) A FORMULATION OF INSULIN BASED ON CRYSTAL-SEEDING IN HYDROGELS AND METHOD THEREOF

(71) Applicant: LAMARK BIOTECH PVT LIMITED, Mahesana (IN)

(72) Inventor: Vaibhav BHATIA, Mahesana (IN)

(21) Appl. No.: 17/635,401

(22) PCT Filed: Aug. 7, 2020
(86) PCT No.: PCT/IB2020/057466

§ 371 Date: Feb. 15, 2022

(30) Foreign Application Priority Data
Aug. 16, 2019 (IN) 201941033024

(51) Int. Cl. C07K 14/62; A61K 47/10; A61K 47/36

(52) U.S. Cl. CPC C07K 14/62; A61K 47/10; A61K 47/36; A61K 38/00

(57) ABSTRACT

The invention relates to a formulation of insulin hydrogel and also a method for the production of protein microcrystals of desired size in the range between 5 to 15 microns (±10 to ±20%) grown by seeding technique. The formulation comprises insulin in hydrogel. The method of preparation comprises mixing supersaturated solution containing the crystalline seeds of the active ingredient, the protein, with solution comprising the protein, the precipitant and a solution comprising a gelling agent at a temperature that maintains the mixture in the metastability zone. The solution is then kept at said temperature for the required time to allow crystal growth without occurring new nucleation events. The method of preparation of insulin hydrogel using seeding protocol is useful for large scale production, while allowing a higher degree of control over final size of crystals.

### TECHNICAL FIELD OF THE INVENTION

The present invention relates to protein crystallization. More particularly, the invention relates to a formulation of insulin microcrystals and a method of preparing homogeneous batches of insulin microcrystals of desired size in the range between 5-15 microns (±10%, ±20%) with a narrow size distribution by seeding crystals in hydrogels. The method of the invention creates insulin microcrystals for drug delivery in which both stability and release are controlled by the incorporated hydrogel.

### BACKGROUND OF THE INVENTION

Protein crystallization is a process of formation of a protein crystal, which is useful in the study of protein structures for further use in various applications. In the process of protein crystallization, proteins are dissolved in an buffered aqueous solution and mixed with a precipitant solution in order to reach the supersaturated state.

Protein crystallization requires highly purified protein in relatively large quantities, i.e. in a few milligrams. For successful crystallization the sample must have high purity, homogeneity and low polydispersity i.e. single aggregation state. Purity is determined by standard laboratory techniques. Methods such as dynamic light scattering (DLS) or Small-Angle X-ray Scattering (SAXS) are used to detect aggregates or polydispersity. Multiple factors influence the whole process of protein crystallization, and therefore, a multitude of methods, strategies and techniques have been developed to attain success. However, in most cases, the optimal conditions to obtain crystals of a particular protein is found serendipitously. One emerging strategy in this field employs the use of hydrogels as a media-modifier for the growth of protein crystals. It has been demonstrated that the use of conventional macromolecular hydrogels such as agarose, polyacrylamide, silica and sephadex exhibits direct impact on the formation of protein crystals and their quality. Indeed, crystals of exceptional size and quality are obtained within hydrogels when compared with other traditional crystallization techniques.

Insulin, a hormone produced by beta cells of pancreas promotes the absorption of carbohydrates, especially glucose from the blood into liver, fat and skeletal muscle cells. In these tissues, the absorbed glucose is converted into either glycogen via glycogenesis or fats via lipogenesis, or, in the case of the liver, into both. High concentration of insulin in the blood strongly inhibits glucose production and secretion by the liver. Circulating insulin also affects the synthesis of proteins in a wide variety of tissues. Beta cells are sensitive to glucose concentrations, also known as blood sugar levels. When blood glucose level is high, the beta cells secrete insulin into the blood, whereas when glucose levels are low, the secretion of insulin is inhibited.

Insulin is available in various compositions with varied actions. The action of insulin varies from rapid-acting insulins, which starts acting almost immediately after being injected to long-acting insulins, which keeps working for up to a day and some last even longer.

Pharmaceutically active protein crystals grown in-situ within the hydrogel, provide many significant advantages such as greater stability and controlled liberation. However, different methods used in state of the art uses hydrogels as media or carriers for the growth of protein crystals within the hydrogel resulted in a wide range of crystal sizes. Such a wide range of sizes might be useful to carry out X-ray diffraction characterization but are not useful for clinical applications. Crystals compatible for clinical applications require a smaller size preferably less than 10 microns, with a narrow size distribution preferably less than 20% of the size, to guaranty a more homogeneous and physiological response. The smaller crystal size is also critical to prevent activation of immune response.

The PCT Application “PCT/DK1999/000371” discloses a method of producing seeding microcrystals for the production of human insulin. The microcrystals being free of non-human pancreatic insulin, the method comprises providing an unseeded suspension of human insulin and homogenizing insulin suspension under pressure to result in human insulin microcrystals suitable for use as seeding microcrystals for the production of zinc insulin products. The method of homogenization under pressure also be used for the production of seeding microcrystals for other peptides and proteins, in particular pharmaceutical peptides or proteins such as insulin, GLP-1, glucagon and growth hormones.

The PCT Application “PCT/EP2017/060842” discloses a process to manufacture a composition of hydrogel comprising pharmaceutically active protein crystals grown in-situ within the hydrogel, wherein the pharmaceutically active protein crystals grown in-situ have an average size of less than 20 microns, which comprises sequentially blending a pharmaceutically active protein solution in their buffer composition by using a value of supersaturation in the metastable zone width (MSZW) or nearby the super solubility curve, with a precipitant solution, wherein acetone is absent, and a gelator solution and inducing the nucleation by lowering the temperature and finally storing the resulting mixture at a constant temperature preferably at room temperature at 1 atm.

The PCT Application “PCT/FI2005/000011” discloses a crystallizing method for macromolecules, especially proteins and polypeptides, in which selected polysaccharides of biological origin, such as alginate, pectin, dextrin or chitosan and hydrolysates are used as reagents. Sedimentation of the crystals is prevented thus the uniformity of the product contributed with the method. The method is useful to prepare new crystal forms of the polypeptide and to improve the stability of crystals.

The Japanese Application “JP5352596B2” discloses an injectable insulin formulation, which is capable of modifying the amount of insulin released based on the patient's tissue glucose levels and also the methods for preparation of the formulation. The formulation is administered via subcutaneous, intradermal or intramuscular administration. The formulations contain insulin, an oxidizing agent or enzyme and a reducing agent or enzyme, a diluent and optionally one or more thickening agents. If a thickening agent is present in the formulation, the thickening agent increases the viscosity of the formulation following administration. The insulin is released from the formulation as a function of the patient's tissue glucose level, which in turn maintains the patient's blood glucose level within an optimum range. The formulation is also designed to release insulin into the systemic circulation over time with a basal release profile following injection in a patient.

Generally, homogeneity and size of crystals are of paramount relevance for clinical application of solid crystalline suspensions. On one hand, the size of the crystals has to allow the subcutaneous injection with thin needles. On the other hand, it has to meet the dissolution rate established to keep a fixed delivery rate. In the available methods of preparation, it is critical to control the size of the particle because of the intrinsic nucleation behavior.

Hence, there is still a need to provide a methodology that allows the production of protein crystals, which are directly useful in treatment.

### SUMMARY OF THE INVENTION

The invention relates to a formulation of insulin hydrogel and also a method for the production of protein microcrystals grown by a seeding technique within a hydrogel with desired size in the range between 5-15 microns (±10%, ±20%) with a narrow size distribution, which is compatible for therapeutic applications. The described method is based on the seeding in batch crystallization in a hydrogel media, which is compatible with scaling-up.

The formulation of the invention comprises insulin in the hydrogel, which is either macromolecular or supramolecular in nature. The macromolecular hydrogel comprises any of the compound such as agarose, gelatin, carrageenan, Poly (Ethylene Glycol) (PEG). The agarose gel comprises polysaccharides of agarobiose units, wherein agarobiose is a disaccharide formed by the union of D-galactose and 3,6-anhydro-L-galactose. The PEG hydrogel is composed of poly (ethylene glycol) monomethyl ether monomethacrylate (PEGMA) of average molecular weight (MW) 1100 Da cross-linked with poly (ethylene glycol) dimethacrylate (PEGDMA) of MW 1200 Da.

The supramolecular peptide-based hydrogel is a Fmoc dipeptide and is selected from Fmoc-FF or Fmoc-AA. The Fmoc-FF hydrogel is based on the dipeptide N-(9-Fluorenylmethoxycarbonyl)-L-phenylalanine-L-phenylalanine and the Fmoc-AA hydrogel is based on the dipeptide N-(9-Fluorenylmethoxycarbonyl)-L-alanine-L-alanine.

The invention further discloses the method of preparation of homogeneous batches of insulin microcrystals of desired size in the range between 5-15 microns (±10%, ±20%) with a narrow size distribution by seeding crystals in hydrogels. The method of preparation comprises mixing supersaturated solution containing the crystalline seeds of the active ingredient, the protein, with a solution comprising the protein, the precipitant and a solution comprising a gelling agent at a temperature that maintains the mixture in the metastability zone. The solution is then maintained at room temperature for the required time to allow growth of the crystals without occurring new nucleation events. In the invention, the seeding protocol, which is based on the growth of the seeds in the hydrogel media produce the number of crystals coincident with the number of seeds used.

The method of preparation of insulin hydrogel using seeding protocol is useful and feasible for large-scale production, allows a higher degree of control over the number and final size of crystals of 5-15 microns which is critical for therapeutic applications. The invention allows to use a fixed number of seeds to grow to the desired crystals sizes.

### BRIEF DESCRIPTION OF DRAWINGS

FIG. 1 illustrates a method for production of formulation of a hydrogel comprising a pharmaceutically active protein crystals grown by a seeding technique.

FIG. 2a illustrates the phase diagram for the crystallization of macromolecules.

FIG. 2b illustrates the schematic representation of a protein crystallization phase diagram.

FIG. 3a illustrates the phase diagram for the crystallization of macromolecules showing the trajectory of the seeding protocol metabolic zone of the solution.

FIG. 3b illustrates the diagrammatic representation of the size distribution of the macromolecules.

FIG. 4a illustrates the optical microscopy of insulin crystals obtained by the seeding technique in hydrogel.

FIG. 4b illustrates the scanning electron microscopy image of the same sample as of FIG. 4a, showing the narrow size distribution.

### DETAILED DESCRIPTION OF THE INVENTION

In order to more clearly and concisely describe and point out the subject matter of the claimed invention, the following definitions are provided for specific terms, which are used in the following written description.

The term “Protein Crystallization” refers to a process of formation of a protein crystal, which are highly ordered microscopic structures of the constituent protein and are useful to determine three-dimensional structure as well as various other biotechnological applications.

The term “Hydrogel” refers to a network of polymer chains that are hydrophilic found as a colloidal gel in which water is the dispersion medium.

The term “Seeding” refers to the growth of protein seed crystals in the metastable region of the phase diagram.

The term “Solubility” is defined as the concentration of protein in the solution that is in equilibrium with the protein in crystalline state.

The term “Supersolubility Curve” is defined as the line separating conditions under which spontaneous nucleation (or phase separation or precipitation) occurs from those under which the crystallization solution remains clear if left undisturbed.

The term “Labile region” is interpreted as the supersaturated region in which both nucleation and growth might be expected to occur.

The term “Precipitation region” is understood as the zone of the phase diagram above the labile zone in which only amorphous (non-crystalline) material is obtained.

The term “Metastability zone” is understood as the region of the phase diagram where nuclei develops into crystals without any nucleation.

The term “Region of sub-saturation or under-saturation” is understood as zone of the phase diagram in which the protein concentration is lower that the solubility.

The term “Precipitant” is understood as the compound or mixture of compounds that provokes the precipitation of the protein in solution when the concentrations are above the solubility curve.

The term “Gelling agent” is a compound capable of forming a hydrogel.

The term “Average size” is understood as sum of the lengths of all the measured crystals divided by the number of measured lengths.

The term “Crystals-lengths” are defined as the two major dimensions of the rhombohedral insulin-crystal.

The term “Fmoc-FF” is the dipeptide N-(9-Fluorenylmethoxycarbonyl)-L-phenylalanine-L-phenylalanine, terminated by a free carboxyl group at their C-terminus end.

The term “Fmoc-AA” is the dipeptide N-(9-Fluorenylmethoxycarbonyl)-L-alanine-L-alanine.

The invention relates to a formulation of insulin hydrogel and also a method for the production of microcrystals of desired size in the range between 5-microns (±10%, ±20%) with a narrow size distribution to be used directly in therapeutic treatment. The described method is based on the seeding in batch crystallization method in hydrogel media and is therefore scalable.

The formulation of the invention comprises insulin in hydrogel. The pharmaceutically active protein crystals grown by a seeding protocol within the hydrogel.

The hydrogel is a viscoelastic solid-like material comprising an elastic cross-linked network and water, wherein water is the major component and the pharmaceutically active protein crystals are composite materials of the protein in crystalline state having an average size of 5 to 15 microns (±10 to ±20%) microns of size as measured by scanning electronic microscopy.

Further, the hydrogel used in the invention is macromolecular or supramolecular in nature. The macromolecular hydrogel comprises any of the compounds such as agarose, gelatin, carrageenan, poly (ethylene glycol). Preferably, the agarose gel comprises polysaccharides of agarobiose units; wherein agarobiose is a disaccharide formed by the union of D-galactose and 3,6-anhydro-L-galactose. Also preferably, the PEG hydrogel is composed of poly (ethylene glycol) monomethyl ether monomethacrylate of average molecular weight (MW) 1100 Da cross-linked with poly (ethylene glycol) dimethacrylate (PEGDMA) of MW 1200 Da.

The supramolecular peptide-based hydrogel is a Fmoc dipeptide and more preferably Fmoc-FF and Fmoc-AA. The Fmoc-FF hydrogel is based on the dipeptide N-(9-Fluorenylmethoxycarbonyl)-L-phenylalanine-L-phenylalanine, and the Fmoc-AA hydrogel is based on the dipeptide N-(9-Fluorenylmethoxycarbonyl)-L-alanine-L-alanine.

The pharmaceutically active protein crystals are not only restricted to insulin but also selected from lysozyme, albumins, phosphatase alkaline, glucose isomerase, growth hormone, somatotropin, factor VIII, factor IX, antithrombin III, immunoglobulins, erythropoietin, interferons, papain, trypsin, hyaluronan-degrading enzymes, collagenase, streptokinase, glucagon, thyrotropin, secretin, humanized chimeric monoclonal antibodies (mAB), vaccines, Human Immunodeficiency Virus (HIV) antigens and hepatitis C antigens.

More preferably, the pharmaceutically active protein crystals are insulin crystals, and the hydrogel is a peptide-based hydrogel formed by Fmoc-AA or an agarose gel comprising polysaccharides of agarobiose units. More preferably, the pharmaceutically active protein crystals are insulin crystals, and the hydrogel is formed by PEG with an average MW 1100 Da.

The invention further discloses the method of preparation of homogeneous batches of insulin microcrystals of desired size in the range between 5-15 microns (±10%, ±20%) with a narrow size distribution by seeding crystals in hydrogels. The method of preparation comprises mixing the supersaturated solution containing the crystalline seeds of the active ingredient, the protein, with a solution comprising the protein, the precipitant and a solution comprising a gelling agent at a temperature that maintains the mixture in the metastability zone. The solution is then maintained at room temperature for the required time to allow crystal growth without occurring new nucleation events.

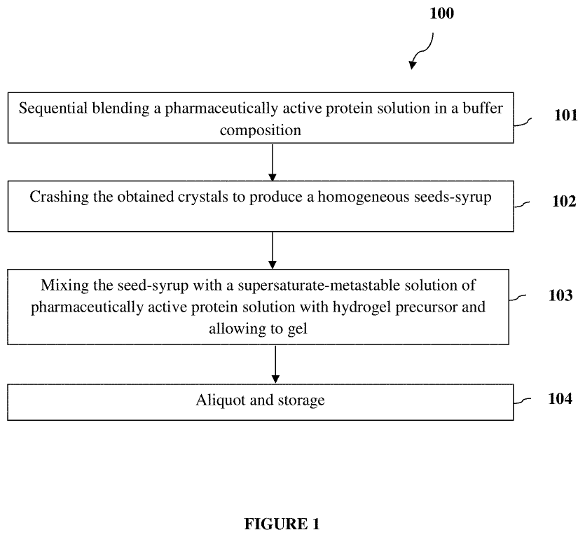

FIG. 1 illustrates the method for preparation of formulation of a hydrogel comprising a pharmaceutically active protein crystals grown by a seeding technique. The method (100) of the invention starts with a step (101) of sequentially blending a pharmaceutically active protein solution in an appropriate buffer composition. This blending is achieved by using a value of supersaturation in the supersaturated zone width or nearby the super solubility curve with a precipitant solution, wherein acetone is absent, to form a crystalline material. The pharmaceutically active protein is insulin. The precipitate solution comprises Hydrochloric acid (HCl), Zinc Chloride (ZnCl₂) and sodium citrate. The concentration of HCl used is in the range between 5 mM and 20 mM, the concentration of ZnCl₂ used is at a concentration of 5 mM and the concentration of sodium citrate used is in the range between 15 mM and 50 mM. At step (102), the obtained crystals regardless of size and shape are crashed to produce a seed-syrup of homogenous size by grinding and/or sieving. At step (103), the specific volume of the homogeneous seed-syrup is mixed with a specific volume of a supersaturate solution of insulin containing the hydrogel precursor and allowed it to gel. The gelator solution is the Fmoc-AA hydrogel or agarose hydrogel. At step (104), the resulting hydrogel mixture is stored at room temperature and at an atmospheric pressure at 101325 Pa i.e. at 1 atmospheric pressure.

In the invention, the seeding protocol, which is based on the growth of the seeds in the hydrogel media produce the number of crystals coincident with the number of seeds used.

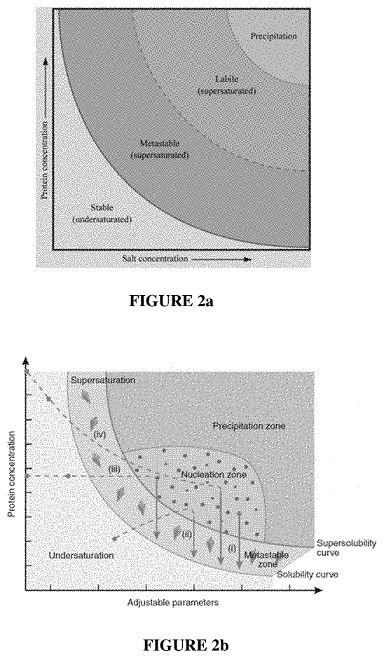

FIG. 2a illustrates the phase diagram for the crystallization of macromolecules. The solubility diagram is divided sharply into a region of undersaturation and a region of supersaturation by the line denoting maximum solubility at specific concentrations of a precipitant, which is a salt or a polymer. The line represents the equilibrium between the existence of the solid phase and the free-molecule phase in solution. The region of supersaturation is further divided in a more uncertain way into the metastable and labile regions. In the metastable region, nuclei develop into crystals without any nucleation whereas in the labile region nuclei develop into crystals with nucleation. The final region, at very high supersaturation, is denoted as the precipitation region, where this result might be most probable. The crystals are only be grown from a supersaturated solution and creating such a solution supersaturated in the protein of interest is the immediate objective in growing protein crystals.

FIG. 2b illustrates the schematic representation of a protein crystallization phase diagram. The adjustable parameters include precipitant or an additive concentration, pH and temperature. The four major crystallization methods are represented as (i) micro batch, (ii) vapor diffusion, (iii) dialysis and (iv) Free Interface Diffusion (FID). Each method involves a different route to reach the nucleation and metastable zones, assuming the adjustable parameter is the precipitant concentration. The filled black circles represent the starting conditions and the two alternative starting points are shown for FID and dialysis because the under-saturated protein solution contains either protein alone or protein mixed with a low concentration of the precipitating agents.

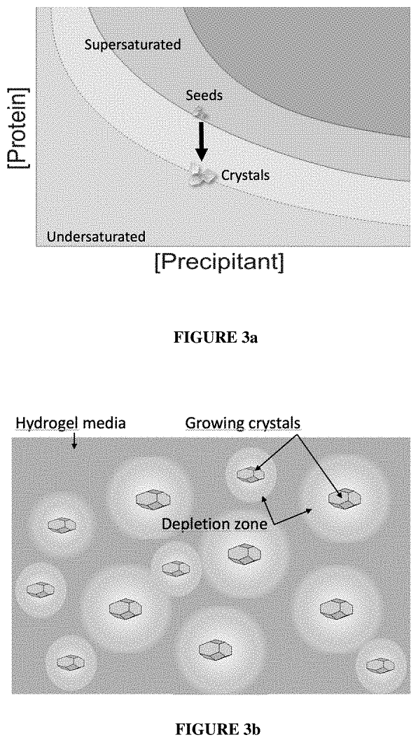

FIG. 3a illustrates the phase diagram for the crystallization of macromolecules showing the trajectory of the seeding protocol metabolic zone of the solution. The supersaturated solution containing the crystalline seeds of the active ingredient insulin is mixed with a solution comprising the protein, the precipitant and a solution comprising a gelling agent such as Fmoc-AA hydrogel or agarose hydrogel at a temperature that keeps the mix in the metastability zone as illustrated in the figure. The solution is then maintained at said temperature for the required time to allow crystal growth without occurring any new nucleation events. The FIG. 3a illustrates the formation of crystals.

FIG. 3b illustrates the diagrammatic representation of the size distribution of the macromolecules. The crystal growth process creates a protein depletion zone as illustrated in the FIG. 3b around the growing crystals, which permits the growth under a homogeneous environment. The method depicts the efficient manner to control the growth path of crystal by controlling the concentration of insulin in the hydrogel. Importantly, the hydrogel provides a homogeneous media for the diffusion-based continuous growth and does not require stirring and the absence of stirring makes the method more compatible to scale-up for industrial production. The stirring-free method further prevents frothing, which causes loss of protein by denaturation at the liquid-air interface while reducing the cost of protein production and crystallization.

Having generally described in this invention, a further understanding can be obtained by reference to certain specific examples, which are provided herein for purposes of illustration only and are not intended to be limiting unless otherwise specified.

### Example 1: A Method of Preparation of Insulin in Agarose Hydrogel

The formulation of insulin protein in agarose gel is prepared by sequentially dissolving insulin to a final concentration of 5 mg/mL, in 6 mM HCl, 5 mM ZnCl₂, 16 mM sodium citrate at pH 7.0 and 0.04% agarose. The resulting crystals are stored at 20° C. at 1 atm and used to prepare seed-syrup. The syrup, with homogenously sized crystals for seeding, is produced by grinding and/or sieving. A master stock solution of seed syrup, with defined size and concentration is prepared and stored at 20° C. at 1 atm. 1% v/v of seed suspension is then incorporated to the previous composition before adding the hydrogel. The samples are incubated for 24 hours in an incubator at 20° C.

The agarose hydrogel is prepared by dissolving 0.08 grams of disaccharide in 10 mL of water at a final concentration of 0.8% and heating to boiling in order to completely dissolve the solution and maintained at 45° C. until it is mixed with the rest of the components.

### Example 2: A Method of Preparation of Insulin in Fmoc-AA Hydrogel

Human insulin in 20 mM HCl at 5 mg/mL final concentration is mixed with 5.0 mM ZnCl2, 22.0 mM sodium citrate at pH 7.0 and completed with water to the final volume. The resulting crystals are stored at 20° C. at 1 atm and used to prepare the seed-syrup. The syrup, with homogenously sized crystals for seeding, is produced by grinding and/or sieving. A master stock solution of seed syrup with defined size and concentration is made and stored at 20° C. at 1 atm. Fmoc-AA, dissolved in DMSO is added as the last step to produce the hydrogel, resulting in a final concentration of 0.25% w/v of Fmoc-AA and 5% of DMSO.1% v/v of seed suspension is then incorporated to the previous composition before adding the hydrogel. Finally, the samples are incubated for 24 hours in an incubator at 20° C.

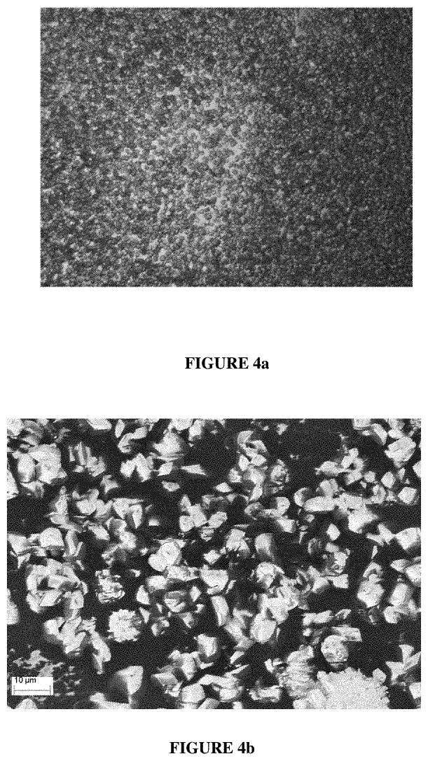

FIGS. 4a and 4b illustrates the optical and scanning electron microscopy of insulin crystals obtained by the seeding technique in hydrogel. The FIGS. 4a and 4b shows the crystals of approximately 5 to 15 microns (±10 to ±20%) with a narrow size distribution. The crystallization process is carried out using the seeding method in hydrogel media at atmospheric pressure. As denoted in the examples, the protein seed crystals, mixture of precipitant and gelling agent are sequentially mixed and the solution is placed in a supersaturation value in the metastability zone width, which allows only crystals growth as depicted in the figure.

The invention discloses a formulation of insulin hydrogel and the method of preparation of the insulin hydrogel by seeding protocol. The formulation is useful in the controlled liberation of the pharmaceutically active protein crystals. The insulin crystals are useful in the treatment of type I and type II diabetes.

The formulation prepared by the seeding technique results in the formation of insulin microcrystals of desired size in the range between 5-15 microns (±10%, ±20%) with a narrow size distribution suitable for effective drug delivery in which both stability and release are controlled by the incorporated hydrogel.

The method of preparation of the insulin hydrogel using seeding protocol is useful and feasible for large-scale production, allows a higher degree of control over the number and final size of crystals, which is critical for therapeutic application. The invention also allows to use a fixed number of seeds to grow to the desired crystals sizes. The method defines the efficient control of the growth path of crystal by controlling the concentration of insulin in the hydrogel. Further, the absence of stirring makes the method more compatible to scale-up for industrial production. The stirring-free method prevents frothing, which causes loss of protein by denaturation at the liquid-air interface while reducing the cost of protein production and crystallization.

We claim:

1. A method of preparation of hydrogel formulation comprising a pharmaceutically active protein crystals, grown by a seeding technique, the method (100) comprises the steps of:
a. sequential blending a pharmaceutically active protein solution in a buffer composition (101);
b. crashing the obtained crystals to produce a homogeneous seeds-syrup (102);
c. mixing the specific amount of the homogeneous seed-syrup with a specific volume of a supersaturate solution of pharmaceutically active protein solution containing the hydrogel precursor and allowing to gel (103); and
d. storing the resulting hydrogel mixture at room temperature and at an atmospheric pressure at 101325 Pa i.e. at 1 atmospheric pressure (104).

2. The method as claimed in claim 1, wherein the pharmaceutically active protein is insulin.

3. The method as claimed in claim 1, wherein the precipitate solution comprises Hydrochloric acid (HCl) in the range between 5 nM and 20 mM, Zinc Chloride (ZnCl2) at a concentration of 5 mM and sodium citrate in the range between 15 mM and 50 mM.

4. The method as claimed in claim 1, wherein the seeding technique results in formation homogeneous suspension of microcrystals of size in the range between 5 to 15 microns (±10 to ±20%) with narrow size distribution.

5. A formulation of hydrogel, the formulation comprises a pharmaceutically active protein crystal at a concentration of 3 mg/mL to 15 mg/mL, wherein active protein microcrystal is insulin with an average size in the range between 5 to 15 microns (±10 to ±20%).

6. The formulation as claimed in claim 5, wherein the hydrogel is macromolecular and made up of compound selected from the group consisting of agarose, gelatin, carrageenan or Poly (Ethylene Glycol) (PEG).

7. The formulation as claimed in claim 5, wherein the hydrogel is supramolecular and made up of Fmoc-FF or Fmoc-AA.

8. The formulation as claimed in claim 5, wherein the Fmoc-FF hydrogel is based on the dipeptide N-(9-Fluorenylmethoxycarbonyl)-L-phenylalanine-L-phenylalanine and the Fmoc-AA hydrogel is based on the dipeptide N-(9-Fluorenylmethoxycarbonyl)-L-alanine-L-alanine.

9. The formulation as claimed in claim 5, wherein agarose hydrogel comprises polysaccharides of agarobiose units, wherein agarobiose is a disaccharide formed by the union of D-galactose and 3,6-anhydro-L-galactose.

10. The formulation as claimed in claim 5, wherein PEG hydrogel comprises poly (ethylene glycol) monomethyl ether monomethacrylate (PEGMA) of average molecular weight of 1100 Da cross-linked with poly (ethylene glycol) dimethacrylate (PEGDMA) of MW 1200 Da.
